(19) United States
(12) Patent Application Publication
(10) Pub. No.: US 2016/0051495 A1
(43) Pub. Date: Feb. 25, 2016

(54) Oral Transmucosal Compositions including C-SERMs for Low Testosterone Levels in Men

(71) Applicants: Tsu-I Catherine Wang, Sugar Land, TX (US); Bruce Vincent Biundo, Houston, TX (US)

(72) Inventors: Tsu-I Catherine Wang, Sugar Land, TX (US); Bruce Vincent Biundo, Houston, TX (US)

(73) Assignee: PROFESSIONAL COMPOUNDING CENTERS OF AMERICA, Houston, TX (US)

(21) Appl. No.: 14/591,748

(22) Filed: Jan. 7, 2015

Related U.S. Application Data
(60) Provisional application No. 62/039,810, filed on Aug. 20, 2014.

(51) Int. Cl. A61K 31/138; A61K 47/10; A61K 47/02; A61K 47/38; A61K 47/42; A61K 47/36

(52) U.S. Cl. CPC A61K 31/138; A61K 47/42; A61K 47/36; A61K 47/02; A61K 47/38; A61K 47/10

(57) ABSTRACT

Formulations for oral transmucosal compositions that include clomiphene-like selective estrogen receptor modulators (C-SERMs) in combination with transmucosal absorption enhancers are disclosed. Oral transmucosal compositions can be for fast release or slow release, and can be administered to increase bloodstream testosterone levels and thereby reduce symptoms of testosterone deficiency. Oral transmucosal compositions include liquid dosage forms, solid dosage forms, and chewing gums. Further dosage forms include mucoadhesive thin strips, thin films, tablets, patches, and tapes, among others. Other dosage forms are: mucoadhesive liquids such as gel-forming liquid; gel-forming; semisolids; and gel-forming powders, among other dosage forms that exhibit mucoadhesive properties, and provide oral transmucosal delivery of C-SERMs. Oral transmucosal compositions allow the delivery of C-SERMs directly into the patient's bloodstream, thereby providing high bioavailability of C-SERMs; therefore, required dose is lower.

### CROSS-REFERENCE TO RELATED APPLICATIONS

This application claims the benefit of U.S. Provisional Application Ser. No. 62/039,810, filed Aug. 20, 2014, which is hereby incorporated by reference.

### BACKGROUND

1. Field of the Disclosure

The present disclosure relates generally to pharmaceutical compositions, and more particularly, to oral transmucosal compositions including clomiphene-like selective estrogen receptor modulators (C-SERMs) for testosterone deficiency.

2. Background Information

Male testosterone deficiency is a syndrome associated with hormonal profile changes which negatively affects libido, sexual function, mood, behavior, lean body mass, and bone density. Actually, testosterone deficiency has been related to low quality of erections, loss of libido, osteoporosis, weight gain, muscle weakness, decreased lean body mass, diabetes mellitus, and cognitive changes. The decrease in serum testosterone levels may be due to primary testicular failure and/or dysfunction of the hypothalamic-pituitary axis. This testosterone deficiency in aging males is associated with increased body weight, adipose tissue, and changes in estrogen levels due to peripheral conversion of testosterone to estradiol. The negative feedback mechanism from excess estradiol results in a paradoxically low luteinizing hormone (LH) secretion from the pituitary despite a physiologically low testosterone level.

Currently, the most common treatment for symptomatic male testosterone deficiency is testosterone therapy with various transdermal, oral, buccal, and injectable delivery methods. These methods typically involve very high doses of testosterone. The main purpose of the testosterone replacement therapy is to achieve normal range of testosterone serum levels.

Testosterone replacement therapy can be associated with side effects such as gynecomastia and nipple tenderness. Most importantly, long term testosterone replacement therapy will cause testicular atrophy and decline in sperm counts due to suppression of the hypothalamic-pituitary-gonadal axis via a negative feedback mechanism. Physiologic inhibition of pituitary gonadotropin secretion in men by testosterone is mainly mediated by its aromatization to estradiol which inhibits hypothalamic secretion of gonadotropin releasing hormone (GnRH). Low levels of GnRH further decrease production of LH and follicle stimulating hormone (FSH) by the pituitary gland. The low LH levels translate to low testosterone production by the Leydig cells in the testes. The reduction in FSH could result in suppression of spermatogenesis.

Selective Estrogen Receptor Modulators (SERMs) are structurally unique compounds that interact with intracellular estrogen receptors in target organs. This class of agents can have either antagonist or agonist properties, and in certain cases, both properties. SERMs such as tamoxifen and raloxifene display unusual pharmacological effects with estrogen agonist properties in some tissues (bone, liver and cardiovascular system), estrogen antagonist properties in other tissues (brain and breast), and mixed agonist/antagonist properties in the uterus. Clomiphene and SERMs that act like clomiphene, acts specifically as an estrogen antagonist in the brain, specifically in the hypothalamus and pituitary sites. Due to the negative feedback mechanism, estradiol slows down the release of GnRH, which results in the reduction of LH/FSH from the pituitary. Clomiphene acts to increase the release of GnRH, LH, and FSH. LH and FSH then act on the testes to increase the production of testosterone and sperm, respectively.

Oral dosage forms usually subject the active pharmaceutical ingredient (API) to degradation in the gastrointestinal tract and the first pass metabolism in the liver, and are commonly associated with a delayed onset. Injections and implanted pellets involve local pain, and the required help of health care professionals makes these dosage forms inconvenient and expensive.

Transdermal administration (e.g., creams, gels, etc.) provides the benefit that the first-pass metabolism and degradation in the gastrointestinal tract are avoided and the treatment is not painful. Unfortunately, transdermal compositions, excluding patches, are often associated with low percentages of absorption through the skin. Another drawback is that a large part of the API remains on the skin exhibiting the potential risk of being transferred to another person through direct skin-to-skin contact. Additionally, the non-absorbed portion of API is lost to the surrounding environment making these formulations non-environmentally-friendly.

Oral transmucosal delivery is a particularly advantageous delivery route. It is a non-invasive drug delivery method with the benefits of better patient compliance, less risk of infection, and lower cost than invasive procedures such as injection and implantation. Oral transmucosal delivery can also provide a much shorter onset time (i.e., the time from administration to therapeutic effect) than oral delivery does. It is simple and can be administered by a caregiver or the patient with minimal discomfort.

Oral transmucosal administration involves the patient holding the compositions in the oral cavity while the API dissolves in the available fluid, diffuses through the mucosa lining of the mouth, and is absorbed directly into the bloodstream bypassing the gastrointestinal tract as well as the first pass hepatic metabolism.

### SUMMARY

The present disclosure refers to oral transmucosal compositions that may include one or more clomiphene-like selective estrogen receptor modulators (C-SERMs) in order to increase testosterone levels in a patient's bloodstream and reduce symptoms of testosterone deficiency. According to some embodiments, the oral transmucosal compositions include different components, such as active pharmaceutical ingredients (APIs), transmucosal absorption enhancers, suitable vehicles, and suitable additives, among others.

According to some embodiments, APIs include C-SERMs such as clomiphene (Clomid®), analogs thereof, or any other chemical known to people skilled in the art that acts on estrogen receptors and blocks the normal estrogen feedback control on the hypothalamus and subsequent negative feedback control on the pituitary.

In some embodiments, the C-SERM employed in oral transmucosal compositions is clomiphene. In one embodiment, clomiphene within oral transmucosal compositions is clomiphene citrate or an analog thereof. In another embodiment, clomiphene within oral transmucosal compositions is zuclomiphene, enclomiphene, or a combination of the two clomiphene isomers.

In some embodiments, various additives are included to facilitate the preparation of suitable dosage forms. For example, additives include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants, among others.

In some embodiments, transmucosal absorption enhancers provide more efficient API skin and mucosal tissue penetration. In these embodiments, the transmucosal absorption enhancers allow lower API dosage requirements.

In some embodiments, amount of absorption enhancers included in oral transmucosal compositions range from about 0.1% to about 20%, most suitable amount is of about 1% to about 10%. These percent ranges may refer to % weight by weight, % weight by volume, or % volume by volume.

In some embodiments, oral transmucosal compositions allow the delivery of C-SERMs directly into the patient's bloodstream bypassing the gastrointestinal tract and the hepatic metabolism. In these embodiments, bypassing the gastrointestinal tract and the hepatic metabolism results in a higher percentage of bioavailability of C-SERMs to the patient. Further to these embodiments, adjustments of C-SERMs dosages may be achieved when using the disclosed oral transmucosal compositions.

In some embodiments, oral transmucosal compositions are administered in the oral cavity at the sublingual, palatal, buccal, gingival, or the like.

In some embodiments, oral transmucosal compositions provide dosage regimens of C-SERMs that are tailored for individual patients. In an example, depending on the baseline serum concentrations of testosterone and estradiol in a patient, a medical doctor may prescribe an oral transmucosal composition with a dosage regimen to more closely mimic the circadian rhythm and physiological pulsatile secretion of testosterone, thereby keeping the testosterone and estradiol levels within suitable ranges.

In some embodiments, oral transmucosal compositions are administered at a dosage range of about 5 mg/day to about 100 mg/day of clomiphene, preferably about 25 mg/day to about 50 mg/day.

In some embodiments, oral transmucosal compositions include liquid dosage forms such as sublingual solutions, emulsions, suspensions, and liquid sprays, among others. In other embodiments, oral transmucosal compositions include solid dosage forms such as sublingual tablets, and buccal troches, among others. In yet other embodiments, oral transmucosal dosage forms include chewing gums.

In some embodiments, oral transmucosal dosage forms include mucoadhesive polymers as part of the compositions. Examples of dosage forms include mucoadhesive thin strips, thin films, tablets, patches, and tapes, among others. In other embodiments, dosage forms include: mucoadhesive liquids such as gel-forming liquid; semisolids such as gels, gel-forming ointments, and gel-forming pastes; gel-forming powders, or any other dosage forms that exhibit mucoadhesive properties and provide oral transmucosal delivery of C-SERMs.

In some embodiments, providing low dose formulations in any of the above identified dosage forms will result in acceptable testosterone levels in the patient. This contrasts with current popular topical treatment options, which use very high dosages of testosterone to get a few milligrams of testosterone absorbed into the bloodstream.

In some embodiments, oral transmucosal dosage forms are designed for fast release and transmucosal absorption of C-SERMs. In other embodiments, oral transmucosal dosage forms are designed for slow release and absorption of C-SERMs over a prolonged period of time.

In some embodiments, the desired testosterone levels may be controlled by adjusting the dosage regimen of C-SERMs.

Numerous other aspects, features, and benefits of the present disclosure may be made apparent from the following detailed description.

### DETAILED DESCRIPTION

The present disclosure is here described. Other embodiments may be used and/or other changes may be made without departing from the spirit or scope of the present disclosure. The illustrative embodiments described in the detailed description are not meant to be limiting of the subject matter presented here.

### DEFINITIONS

As used here, the following terms have the following definitions:

“Active Pharmaceutical Ingredients (APIs)” are chemical compounds that induce a desired effect, and include agents that are therapeutically effective, prophylactically effective, or cosmeceutically effective.

“Absorption Enhancer” or, equivalently, “Penetration Enhancer” is a substance used to modify, generally to increase, the rate of permeation through mucous membrane, skin or other body tissue of one or more substances (e.g., APIs) in a formulation.

“Selective Estrogen Receptor Modulators (SERMs)” are chemical compounds that interact with intracellular estrogen receptors in target organs.

“Clomiphene-like SERMs (C-SERMs)” are chemical compounds that act like clomiphene, as selective estrogen antagonist in the brain, specifically in the hypothalamus and pituitary sites. As such, the C-SERMs act to increase the release of GnRH, LH, and FSH. LH and FSH then act on the testes to increase the production of testosterone and sperm, respectively.

“Treating” and “Treatment” is the reduction in severity and/or frequency of symptoms, elimination of symptoms and/or underlying cause, prevention of the occurrence of symptoms and/or their underlying cause, and improvement or remediation of damage.

“Vehicle” is a substance of no therapeutic value that is used to convey at least one API for administration.

### DESCRIPTION OF THE DISCLOSURE

Embodiments of the present disclosure are directed towards oral transmucosal delivery of active pharmaceutical ingredient (APIs). Oral transmucosal compositions that include one or more clomiphene-like selective estrogen receptor modulators (C-SERMs) as APIs are described. The present disclosure including C-SERMs is proposed to increase testosterone levels in a patient's bloodstream and reduce symptoms of testosterone deficiency.

Estradiol serves as a major mediator of sex steroid-gonadotropin feedback. Thus, the secretion of luteinizing hormone (LH) and follicle-stimulating hormone (FSH) are, to a large extent, modified by agents that affect the activity of estradiol. C-SERMs have the capacity to compete with estradiol for the estrogen receptors at the level of hypothalamus and pituitary, blunt the activity of estradiol, and increase the amount of LH and FSH the body produces. These increased levels correspond with increased production of testosterone and sperm, respectively. Therefore, C-SERMs can be used for both hypogonadism and male infertility.

Formulation

Oral transmucosal compositions include one or more C-SERMs as APIs, transmucosal absorption enhancers, vehicles, and additives, among other suitable ingredients.

According to some embodiments, APIs include C-SERMs such as clomiphene (Clomid®), analogs thereof, or any other chemical known to people skilled in the art that acts on estrogen receptors and blocks the normal estrogen feedback control on the hypothalamus and subsequent negative feedback control on the pituitary.

In some embodiments, the C-SERM employed in oral transmucosal compositions is clomiphene. In one embodiment, clomiphene within oral transmucosal compositions is clomiphene citrate or an analog thereof. In another embodiment, clomiphene within oral transmucosal compositions is zuclomiphene, enclomiphene, or a combination of the two clomiphene isomers.

The list of C-SERMs above is not exhaustive; other compounds described in the art that meet the set requirements can also be considered.

C-SERM, such as clomiphene, works by stimulating a part of the brain (the pituitary gland) that controls production of two hormones key to reproductive health: FSH and LH. Both hormones are also vital to men. FSH stimulates sperm production in the testicles, and LH stimulates testosterone production; therefore, oral transmucosal compositions can be used in treating a wide variety of conditions resulting from testosterone deficiency.

In some embodiments, various additives are included to facilitate the preparation of suitable dosage forms. For example, additives include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants, among others.

In some embodiments, diluents for solid dosage forms include calcium carbonate, dibasic calcium phosphate, tribasic calcium phosphate, calcium sulfate, kaolin, microcrystalline cellulose, and other cellulose derivatives, sodium chloride, starch and starch derivatives, sucrose, dextrose, lactose, and sorbitol, among others.

Binders for solid dosage forms include starch and starch derivatives, gelatin, sucrose, glucose, dextrose, molasses, lactose, natural and synthetic gums, acacia, sodium alginate, extract of Irish Moss, panwar gum, ghatti gum, mucilage of isapol husks, carboxymethylcellulose, methylcellulose, cellulose derivatives, Veegum, polyvinylpyrolidone, and polyethylene glycols, among others.

Disintegrants for solid dosage forms include veegum, agar, bentonite, alginic acid and alginic acid derivatives, guar gum, starch, sodium starch glycolate, other starch derivatives, clays, cellulose, and cellulose derivatives, among others.

Lubricants for solid dosage forms include stearic acid, stearic acid derivatives, stearic acid salts such as magnesium stearate and calcium stearate, talc, hydrogenated vegetables oils, polyethylene glycols, surfactants, and waxes, among others.

Additionally, solid dosage forms of oral transmucosal compositions include: a glidant, such as colloidal silicon dioxide and talc, among others; a sweetening agent, such as sucrose or saccharin, among others; natural or artificial flavors, such as peppermint, methyl salicylate, or orange flavor, among others.

The pH adjusting agents include sodium bicarbonate, magnesium hydroxide, calcium carbonate, dibasic calcium phosphate, tribasic calcium phosphate, sodium bicarbonate, magnesium hydroxide, potassium hydroxide, citric acid, lactic acid, hydrochloric acid, sulfuric acid, phosphoric acid, sodium phosphate monobasic, and sodium phosphate dibasic, among others.

Surfactants include: polysorbates such as polysorbate 20, 40, 60, and 80, among others; sorbitan esters such as sorbitan monolaurate, and sorbitan monopalmitate, sorbitan monooleate, among others; and sodium lauryl sulfate, among others.

Effervescent agents are usually a combination of one or more acids with one or more bases. Acids are selected from citric acid, tartaric acid, and the like. Bases can be sodium bicarbonate or other suitable agents that may react with acids, and produce gas.

In some embodiments, a stabilizing agent is used to stabilize the API for a specific dosage form. In these embodiments, the stabilizing agent used will depend on the API used as well as the other additive ingredients. Any suitable chemical substance may be used as a stabilizing agent. Stabilizing agents are known to those skilled in the art and therefore will not be discussed further herein.

Mucoadhesive polymers include: gums such as acacia, agarose, alginic acid, sodium alginate and other alginic acid derivatives, carrageenan, gelatin, gellan, guar gum, hakea gum, karaya gum, and locust bean gum, among others; chitosan and chitosan derivatives; hyaluronic acid, pectin, and other polysaccharides; gelatin, polyisoprene, polyisobutylene, polyetherurethane, polyvinylalcohol, polyvinylpyrrolidone, polycarbophil, polyethylene oxide polymers, and pullulan, among others. Mucoadhesive polymers also include: cellulose derivatives such as ethyl cellulose, cellulose acetate, hydroxyethyl cellulose, hydroxypropylcellulose, hydroxypropylmethylcellulose, methylcellulose, methylhydroxyethylcellulose, and sodium carboxymethyl cellulose, among others; poly(acrylic acid)-based polymers such as polyacrylates, poly(methylvinylether-co-methacrylic acid), poly(acrylic acid-co-ethylhexylacrylate), poly(acrylic acid-co-acrylamide), poly(acrylic acid-co-butylacrylate), poly(acrylic acid-co-methyl methacrylate), poly(2-hydroxyethyl methacrylate), polymethacrylates, poly(alkylcyanoacrylate) and other cyanoacrylates, poly(isohexycyanoacrylate), poly(isobutylcyanoacrylate), and hydroxyethyl methacrylate, and any other polymer known to a person skilled in the art that exhibits mucoadhesive characters.

Plasticizers for mucoadhesive polymeric dosage forms include pullulan, hydroxypropyl methylcellulose, propylene glycol, glycerol, sorbitol, mannitol, polyethylene glycols (PEG 200, 400, 600, 1000, 1500, 2000), tartaric acid, malic acid, lactic acid, citric acid, and yonkenafil, and any other chemical known to a person skilled in the art that can increase the plasticity of any mucoadhesive polymer.

In some embodiments, transmucosal absorption enhancers provide more efficient API skin and mucosal tissue penetration. In these embodiments, the transmucosal absorption enhancers allow lower API dosage requirements.

Oral transmucosal absorption enhancers include: enzyme inhibitors such as aprotinin and puromycin, among others; chitosan and chitosan derivatives such as chitosan glutamate, trimethyl chitosan, chitosan-4-thioglycolic acid, 5-methyl-pyrrolidine chitosan, and chitosan-4-thio-butylamidine, among others; alpha, beta, and gamma cyclodextrins such as dimethyl cyclodextrin, sulfobutyl cyclodextrin, 2-hydroxypropyl-beta-cyclodextrin, poly-beta-cyclodextrin, and methylated beta-cyclodextrin, among others; bile salts such as sodium deoxycholate, sodium glycocholate, sodium glycodeoxycholate, sodium glycodihydrofusidate, sodium taurocholate, sodium taurodeoxycholate, sodium tauroglycocholate, sodium taurodihydrofusidate, and sodium ursocholate, among others; chelating agents such as sodium EDTA, citric acid, sodium citrate, sodium salicylate, methylsalicylate, methoxysalicylate, and polyacrylates, among others; alcohols such as ethanol and isopropanol, among others; fatty acids and derivatives such as oleic acid, methyloleate, capric acid, neodecanoic acid, elaidic acid, lauric acid, palmitoylcarnitine, cod liver oil extract, mono glycerides and diglycerides of oleic acid and capric acid, lauric acid, sodium laurate, linoleic acid, sodium fusidate, sodium caprate, glyceryl monolaurate, glyceryl monooleate, glyceryl monostearate, sucrose fatty acid esters, and diethylene glycol monoethyl ether, among others; lecithins and phospholipids such as phosphatidylcholine, lysophosphatidyl choline, and didecanoylphophatidylcholine, among others; sulfoxides such as dimethylsulfoxide and decylmethyl sulfoxide, among others; polyols such as glycerin, propylene glycol, propanediol, and polyethylene glycols of various molecular weights, among others; urea and derivatives such as unsaturated cyclic urea, among others; surfactants such as sodium dodecyl sulfate, sodium lauryl sulfate, dioctyl sodium sulfosuccinate, nonylphenoxypolyoxyethylene, polyoxyethylene alkyl ethers, polyoxyethylene-9-lauryl ether, polyoxyethylene 23 lauryl ether, polyoxyethylene-20-cetyl ether, polyethyleneglycol dodecyl ether, polyethylene glycol-8 laurate, glyceryl monolaurate, polyoxyethylene stearates, polysorbates, sorbitan fatty acid esters, polyoxyethylene castor oil derivatives, benzalkonium chloride, cetylpyridinium chloride, and cetyltrimethylammonium bromide, among others. Other oral transmucosal absorption enhancers include alkylglycosides, azone, hyaluronic acid, sodium hyaluronate, glycine chenodeoxycholate, lauroyl macroglycerides, isopropyl myristate, isopropyl palmitate, glutathione, witepsol, menthol, capsaicin, taurine, tocopheryl acetate, lauroyl macroglycerides, linoleoyl polyoxyl-6 glycerides; diethylene glycol monoethyl ether, dextran sulfate, various saponins, poly-l-arginine, and l-lysine, and any other chemical known to a person skilled in the art that exhibits penetration enhancing effect on transmucosal absorption.

In some embodiments, amount of absorption enhancers included in oral transmucosal compositions range from about 0.1% to about 20%, most suitable amount is of about 1% to about 10%. These percent ranges may refer to % weight by weight, % weight by volume, or % volume by volume.

Bases for chewing gum include cellulosic polymer, and acrylic polymer, among others.

In some embodiments, oral transmucosal compositions include pharmaceutical solvents to produce sprays, solutions, emulsions, suspensions, gels, gel-forming liquids, ointments and pastes, among others.

In some embodiments, pharmaceutical solvents for liquid dosage forms of oral transmucosal compositions include water, liquid polyethylene glycols of various molecular weights, ethyl oleate, medium chain triglycerides, isopropyl myristate, isopropyl palmitate, isopropyl stearate, other pharmaceutically acceptable esters of C8-C22 fatty acids and C2-C6 alcohols, mineral oil, and vegetable oils, among others.

C8-C22 fatty acids include fatty acids having from 8 to 22 carbon atoms, such as myristic acid, palmitic acid, stearic acid, arachidic acid, or oleic acid, among others.

C2-C6 alcohols include alcohols having from 2 to 6 carbon atoms, in particular the C2-C5 alcohols as well as the homologues with 6 carbon atoms including diols and triols such as ethanol, propylene glycol, and glycerol, among others.

Examples of vegetable oils include almond oil, peanut oil, sesame oil, sunflower oil, safflower oil, canola oil, corn oil, and olive oil, among others.

In some embodiments, oral transmucosal ointments and pastes include petrolatum, PCCA Plasticized™ base, paraffin wax, various synthetic wax, lanolin, beeswax, carnauba wax, candelila wax, silicones, isopropylesters, polyols, cellulose ethers, among other suitable bases. In addition, ointment bases also include suitable pharmaceutical solvents, such as water, liquid polyethylene glycols of various molecular weights, ethyl oleate, medium chain triglycerides, isopropyl myristate, isopropyl palmitate, isopropyl stearate, and other pharmaceutically acceptable esters of C8-C22 fatty acids and C2-C6 alcohols, mineral oil, and vegetable oils, among others.

Administration

In some embodiments, oral transmucosal compositions allow the delivery of C-SERMs directly into the patient's bloodstream bypassing the gastrointestinal tract and the hepatic metabolism. In these embodiments, bypassing the gastrointestinal tract and the hepatic metabolism results in a higher percentage of bioavailability of C-SERMs to the patient. Further to these embodiments, adjustments of C-SERMs dosages may be achieved when using the disclosed oral transmucosal compositions.

In some embodiments, oral transmucosal compositions are administered in the oral cavity at the sublingual, palatal, buccal, gingival, or the like. Oral transmucosal compositions may be self-administered by the patient or administered by a medical practitioner, such as a physician or nurse.

In some embodiments, oral transmucosal compositions include liquid dosage forms such as sublingual solutions, emulsions, suspensions, and liquid sprays, among others. In other embodiments, oral transmucosal compositions include solid dosage forms such as sublingual tablets, and buccal troches, among others. In yet other embodiments, oral transmucosal dosage forms include chewing gums.

In some embodiments, oral transmucosal dosage forms include mucoadhesive polymers as part of the compositions. Examples of dosage forms include mucoadhesive thin strips, thin films, tablets, patches, and tapes, among others. In other embodiments, dosage forms include: mucoadhesive liquids such as gel-forming liquid; semisolids such as gels, gel-forming ointments, and gel-forming pastes; gel-forming powders, or any other dosage forms that exhibit mucoadhesive properties and provide oral transmucosal delivery of C-SERMs.

In some embodiments, oral transmucosal dosage forms are designed for fast release and transmucosal absorption of C-SERMs. In other embodiments, oral transmucosal dosage forms are designed for slow release and absorption of C-SERMs over a prolonged period of time.

In some embodiments, oral transmucosal compositions are administered in a single administration whereby a certain amount of C-SERM is administered at once. In an example, one puff of a spray solution is administered representing the full desired dose. In other embodiments, oral transmucosal compositions are administered by multiple administrations in one or more sub-doses over a specified period of time. In an example, one, two or more puffs of a smaller dose is administered preferably shortly after one another.

In some embodiments, oral transmucosal compositions provide dosage regimens of C-SERMs that are tailored for individual patients. In an example, depending on the baseline serum concentrations of testosterone and estradiol in a patient, a medical doctor may prescribe an oral transmucosal composition with a dosage regimen to more closely mimic the circadian rhythm and physiological pulsatile secretion of testosterone, thereby keeping the testosterone and estradiol levels within suitable ranges.

In some embodiments, providing low dose formulations in any of the above identified dosage forms will result in acceptable testosterone levels in the patient. This contrasts with current popular topical treatment options, which use very high dosages of testosterone to get a few milligrams of testosterone absorbed into the bloodstream.

In some embodiments, the dosages (e.g., daily) required depend on the type of C-SERM included in the disclosed oral transmucosal compositions. In other words, some C-SERMs are more potent than others, and hence, the dosing can vary among the various C-SERMs used.

In some embodiments, oral transmucosal compositions are administered at a dosage range of about 5 mg/day to about 100 mg/day of clomiphene, preferably about 25 mg/day to about 50 mg/day.

In some embodiments, the desired testosterone levels may be controlled by adjusting the dosage regimen of C-SERMs.

The following examples are intended to illustrate the scope of the disclosure and are not intended to be limiting. It is to be understood that other pharmaceutical formulations known to those skilled in the art may alternatively be used.

### Examples

Exemplary dosage forms of the oral transmucosal compositions are described below.

Example #1 illustrates formula for one clomiphene citrate sublingual tablet:

| Ingredient | Composition |
|---|---|
| Clomiphene citrate | 25 mg |
| Penetration enhancer(s) | 1-10% |
| Flavor(s) | 0.5-5% |
| Lactose/sucrose (80:20) | q.s. 150-200 mg |

Example #2 illustrates formula for one dose of clomiphene citrate sublingual drops:

| Ingredient | Composition |
|---|---|
| Clomiphene citrate | 25 mg |
| Co-solvent(s) | 10-50% |
| Penetration enhancer(s) | 1-10% |
| Flavor(s) | 0.5-5% |
| Sweetener(s) | 0.1-1.5% |
| Base Solvent | q.s. 0.2 mL |

Example #3 illustrates formula for one dose of clomiphene citrate oral adhesive paste:

| Ingredient | Composition |
|---|---|
| Clomiphene citrate | 25 mg |
| Gelatin | 1-5% |
| Pectin | 1-5% |
| Sodium | 1-10% |
| Carboxymethylcellulose | |
| Xanthan gum | 0.1-5% |
| PEG-90M | 1-10% |
| Penetration enhancer(s) | 1-10% |
| Flavor(s) | 0.5-5% |
| Sweetener(s) | 0.1-1.5% |
| PCCA Plasticized ™ Base* | q.s. 0.2-0.5 mL |

*It is a proprietary gel base produced by Professional Compounding Centers of America (PCCA)

While various aspects and embodiments have been disclosed, other aspects and embodiments are contemplated. The various aspects and embodiments disclosed are for purposes of illustration and are not intended to be limiting, with the true scope and spirit being indicated by the following claims.

1. A pharmaceutical composition comprising one or more clomiphene-like selective estrogen receptor modulators (C-SERMs), wherein the composition is an oral transmucosal formulation that allows delivery of a C-SERM directly into a patient's bloodstream.

2. The pharmaceutical composition of claim 1, wherein the C-SERM is clomiphene or analogs thereof.

3. The pharmaceutical composition of claim 2, wherein the clomiphene is clomiphene citrate or an analog thereof.

4. The pharmaceutical composition of claim 1, wherein the C-SERM is enclomiphene or zuclomiphene.

5. The pharmaceutical composition of claim 1 further comprising an additive selected from the group consisting of include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants.

6. The pharmaceutical composition of claim 5, wherein the diluent comprises calcium carbonate, dibasic calcium phosphate, tribasic calcium phosphate, calcium sulfate, kaolin, microcrystalline cellulose, cellulose derivatives, sodium chloride, starch, starch derivatives, sucrose, dextrose, lactose, or sorbitol.

7. The pharmaceutical composition of claim 5, wherein the binder comprises starch, starch derivatives, gelatin, sucrose, glucose, dextrose, molasses, lactose, natural gums, synthetic gums, acacia, sodium alginate, extract of Irish Moss, panwar gum, ghatti gum, mucilage of isapol husks, carboxymethylcellulose, methylcellulose, cellulose derivatives, Veegum, polyvinylpyrolidone, or polyethylene glycols.

8. The pharmaceutical composition of claim 5, wherein the disintegrant comprises veegum, agar, bentonite, alginic acid, alginic acid derivatives, guar gum, starch, sodium starch glycolate, starch derivatives, clays, cellulose, or cellulose derivatives.

9. The pharmaceutical composition of claim 5, wherein the lubricant comprises stearic acid, stearic acid derivatives, stearic acid salts such as magnesium stearate and calcium stearate, talc, hydrogenated vegetables oils, polyethylene glycols, surfactants, or waxes.

10. The pharmaceutical composition of claim 5, wherein the glidant is colloidal silicon dioxide or talc.

11. The pharmaceutical composition of claim 5, wherein the sweetening agent is sucrose, saccharin, natural flavor, or artificial flavors.

12. The pharmaceutical composition of claim 5, wherein the pH adjusting agent is sodium bicarbonate, magnesium hydroxide, calcium carbonate, dibasic calcium phosphate, tribasic calcium phosphate, sodium bicarbonate, magnesium hydroxide, potassium hydroxide, citric acid, lactic acid, hydrochloric acid, sulfuric acid, phosphoric acid, sodium phosphate monobasic, or sodium phosphate dibasic.

13. The pharmaceutical composition of claim 5, wherein the surfactant comprises a polysorbate, a sorbitan ester, or sodium lauryl sulfate.

14. The pharmaceutical composition of claim 13, wherein the polysorbate is polysorbate 20, polysorbate 40, polysorbate 60, or polysorbate 80.

15. The pharmaceutical composition of claim 13, wherein the sorbitan ester is sorbitan monolaurate, sorbitan monopalmitate, or sorbitan monooleate.

16. The pharmaceutical composition of claim 5, wherein the mucoadhesive polymers comprises gums; chitosan and chitosan derivatives; polysaccharides; gelatin; cellulose derivatives; or poly(acrylic acid)-based polymers.

17. The pharmaceutical composition of claim 16, wherein the gum is selected from the group consisting of acacia, agarose, alginic acid, alginic acid derivatives, carrageenan, gelatin, gellan, guar gum, hakea gum, karaya gum, and locust bean gum.

18. The pharmaceutical composition of claim 16, wherein the cellulose derivative is selected from the group consisting of ethyl cellulose, cellulose acetate, hydroxyethyl cellulose, hydroxypropylcellulose, hydroxypropylmethylcellulose, methylcellulose, methylhydroxyethylcellulose, and sodium carboxymethyl cellulose.

19. The pharmaceutical composition of claim 16, wherein the poly(acrylic acid)-based polymers is selected from the group consisting of polyacrylates, poly(methylvinylether-co-methacrylic acid), poly(acrylic acid-co-ethylhexylacrylate), poly(acrylic acid-co-acrylamide), poly(acrylic acid-co-butylacrylate), poly(acrylic acid-co-methyl methacrylate), poly(2-hydroxyethyl methacrylate), polymethacrylates, poly(alkylcyanoacrylate) and other cyanoacrylates, poly(isohexycyanoacrylate), poly(isobutylcyanoacrylate), and hydroxyethyl methacrylate.

20. The pharmaceutical composition of claim 5, wherein the mucoadhesive polymers comprises hyaluronic acid, pectin, polyisoprene, polyisobutylene, polyetherurethane, polyvinylalcohol, polyvinylpyrrolidone, polycarbophil, polyethylene oxide polymers, or pullulan.

21. The pharmaceutical composition of claim 5, wherein the transmucosal absorption enhancer comprises enzyme inhibitors; chitosan or chitosan derivative; cyclodextrins; bile salts; chelating agents; alcohols; fatty acids and derivatives thereof; lecithins; sulfoxides; polyols; urea and derivatives thereof; surfactants; alkylglycosides, azone, hyaluronic acid, sodium hyaluronate, glycine chenodeoxycholate, lauroyl macroglycerides, isopropyl myristate, isopropyl palmitate, glutathione, witepsol, menthol, capsaicin, taurine, tocopheryl acetate, lauroyl macroglycerides, linoleoyl polyoxyl-6 glycerides; diethylene glycol monoethyl ether, dextran sulfate, saponins, poly-l-arginine, and 1-lysine.

22. The pharmaceutical composition of claim 21, wherein the enzyme inhibitors is aprotinin or puromycin.

23. The pharmaceutical composition of claim 21, wherein chitosan or chitosan derivative is chitosan glutamate, trimethyl chitosan, chitosan-4-thioglycolic acid, 5-methyl-pyrrolidine chitosan, or chitosan-4-thio-butylamidine.

24. The pharmaceutical composition of claim 21, wherein the cyclodextrin is an alpha, beta, or gamma cyclodextrin.

25. The pharmaceutical composition of claim 21, wherein the cyclodextrin is selected from the group consisting of dimethyl cyclodextrin, sulfobutyl cyclodextrin, 2-hydroxypropyl-beta-cyclodextrin, poly-beta-cyclodextrin, and methylated beta-cyclodextrin.

26. The pharmaceutical composition of claim 21, wherein the bile salt is selected from the group consisting of sodium deoxycholate, sodium glycocholate, sodium glycodeoxycholate, sodium glycodihydrofusidate, sodium taurocholate, sodium taurodeoxycholate, sodium tauroglycocholate, sodium taurodihydrofusidate, and sodium ursocholate.

27. The pharmaceutical composition of claim 21, wherein the chelating agent is selected from the group consisting of sodium EDTA, citric acid, sodium citrate, sodium salicylate, methylsalicylate, methoxysalicylate, and polyacrylates.

28. The pharmaceutical composition of claim 21, wherein the alcohol is ethanol or isopropanol.

29. The pharmaceutical composition of claim 21, wherein the fatty acid and derivatives thereof is selected from the group consisting of oleic acid, methyloleate, capric acid, neodecanoic acid, elaidic acid, lauric acid, palmitoylcarnitine, cod liver oil extract, mono glycerides and diglycerides of oleic acid and capric acid, lauric acid, sodium laurate, linoleic acid, sodium fusidate, sodium caprate, glyceryl monolaurate, glyceryl monooleate, glyceryl monostearate, sucrose fatty acid esters, and diethylene glycol monoethyl.

30. The pharmaceutical composition of claim 21, wherein the lecithin is phosphatidylcholine, lysophosphatidyl choline, or didecanoylphophatidylcholine

31.-54. (canceled)
